(19) United States
(12) Patent Application Publication
(10) Pub. No.: US 2013/0245417 A1
(43) Pub. Date: Sep. 19, 2013

(54) SYSTEM AND METHOD FOR DIAGNOSING AND TREATING DISEASE

(71) Applicant: Donald SPECTOR, New York, NY (US)

(72) Inventor: Donald SPECTOR, New York, NY (US)

(21) Appl. No.: 13/438,003

(22) Filed: Apr. 3, 2012

Related U.S. Application Data
(60) Provisional application No. 61/612,575, filed on Mar. 19, 2012.

(51) Int. Cl. A61B 6/00; A61N 5/06

(52) U.S. Cl. 600/407; 607/88

(57) ABSTRACT

A method and system for diagnosing and treating infection or disease, in which an individual takes a photograph of an infected or diseased area of a body using a camera connected to a microprocessor. The photograph is sent to a diagnosing center having a server with a second microprocessor and a database of photographs correlated with different diseases and infections. The second microprocessor scans the image received from the mobile telephone and compares it to the photographs in the database. If a match is found, the second microprocessor then notes the disease or bacteria corresponding to the matching photograph. The second microprocessor then searches the database for a treatment corresponding to the identified disease or bacteria. Once a treatment is identified, information regarding this treatment is automatically sent to the individual's microprocessor.

### CROSS REFERENCE TO RELATED APPLICATIONS

This application claims priority under 35 U.S.C. 119(e) of U.S. Provisional Application No. 61/612,575 filed Mar. 19, 2012.

### BACKGROUND OF THE INVENTION

1. Field of the Invention

The present invention relates to a system and method for diagnosing disease, such as cancer or bacterial infection. The invention relates to a method where an individual takes a high resolution photograph of a wound or diseased area of the body, sends the photograph to a center where it is magnified and enhanced, and software at the center then searches a database containing a library of diseases to find a match. Once a match has been found, the software can send a prescription or other treatment recommendation to a doctor or to an individual for treating the disease or infection.

2. The Prior Art

Photographic databases of diseased cells and bacteria are in use today to help doctors and scientists diagnose illness and infection. In most situations, the scientist or laboratory worker places the cells on a slide under a microscope and takes a photograph of the cells under magnification. These cells can then be compared to the various photographs of similar cells in the database to help diagnose the illness. On example of this can be seen in U.S. Pat. No. 5,911,139 to Jain et al., the disclosure of which is herein incorporated by reference. Jain et al. describes a system for matching a photograph of cells taken under a microscope with photographs in a database.

U.S. Pat. No. 7,756,309 to Gholap et al., the disclosure of which is herein incorporated by reference, also describes a system for indexing and matching medical images in a database.

However, neither of these references teach a method in which in vivo injuries or illnesses can be quickly and remotely diagnosed and treated, without the use of slides and microscopes.

### SUMMARY OF THE INVENTION

It is therefore an object of the invention to provide a system and method for diagnosing illness or infection and providing a treatment to an individual from a remote location.

The present invention comprises a method and system for diagnosing and treating infection or disease, in which an individual takes a photograph of an infected or diseased area of a body using a high definition camera connected to microprocessor and communication device. The camera could be incorporated into a mobile phone that has microprocessing capabilities as well or could be connected to a personal computer with a modem. The camera is equipped to take very high resolution photographs that can be enlarged to a degree where individual cells and bacteria can be discerned. The individual then sends the photograph by text message or email via the microprocessor in the device to a diagnosing center having a server with a microprocessor and a database of photographs correlated with different diseases and infections. The photographs in the database are indexed according to the disease or infection that they show. The photographs in the database show these diseases and infections at the cellular level, so that individual cells, bacteria and other organisms can be seen.

The microprocessor in the server is equipped with software that can scan the image received from the mobile telephone and compare it to the photographs in the database. To cut down on the amount of searching required, the user can specify a particular type of disease, to restrict the search to a particular group of diseases or injuries. This can also be sent in the text message or email with the photograph. The microprocessor then retrieve the categories specified by the user and input them as well as the data from the photograph to search for a match.

The microprocessor is equipped with software that enlarges and enhances the photograph by the individual so that individual cells or bacteria can be seen in the enhanced photograph.

This enlarged and enhanced picture is used by the microprocessor to compare it with the photographs in the database to try to find a match, using visual comparison software. If a match is found, the microprocessor then notes the disease or bacteria corresponding to the matching photograph. The microprocessor then searches the database for a treatment corresponding to the identified disease or bacteria. Once a treatment is identified, information regarding this treatment is automatically sent to the individual's mobile phone or computer.

The present invention provides an instantaneous method for diagnosing and treating injury, illness and infection from a remote location. For example a person suspecting that they might have skin cancer could take a picture of the cancerous lesion and send it to the diagnosing center, who would immediately search for the exact type of cancer, identify it and send back a treatment plan. The photograph when enlarged would show the cancerous skin cells in enough detail to allow the microprocessor to search for a match at the cellular level.

To assist in identification of the disease, a dye can be applied to the infected or diseased area prior to photographing it. In addition, a video could also be taken of the infected or diseased area. The video would show the behavior of the bacteria or microorganisms in the wound. For example, if the specific pathogen cannot be seen, the video could show the rapid deterioration of the cells, thus indicating a certain type of bacterial or viral infection. The database could also contain video recordings of similar infections for comparison as well.

Once a treatment plan has been identified by the microprocessor, it sends the plan to the individual's mobile phone or computer. If the treatment plan involves administering medication, a prescription for the medication can be automatically sent to the mobile phone or personal computer of the user. The prescription could also relate to radiology, chemotherapy or radiation therapy. In another embodiment, the treatment plan could involve administering UV light to a wound to kill infection. In this situation, the microprocessor could send a program application to the individual's mobile phone or personal computer which when activated would generate a beam of the UV light. This could be aimed at the wound to kill the infection.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a schematic drawing of the system according to the invention in use for treating a patient;

FIG. 2 shows a flow diagram of the method steps according to the invention; and

FIG. 3 shows a drawing of a treatment plan according to one embodiment of the invention.

### DETAILED DESCRIPTION OF THE PREFERRED EMBODIMENT

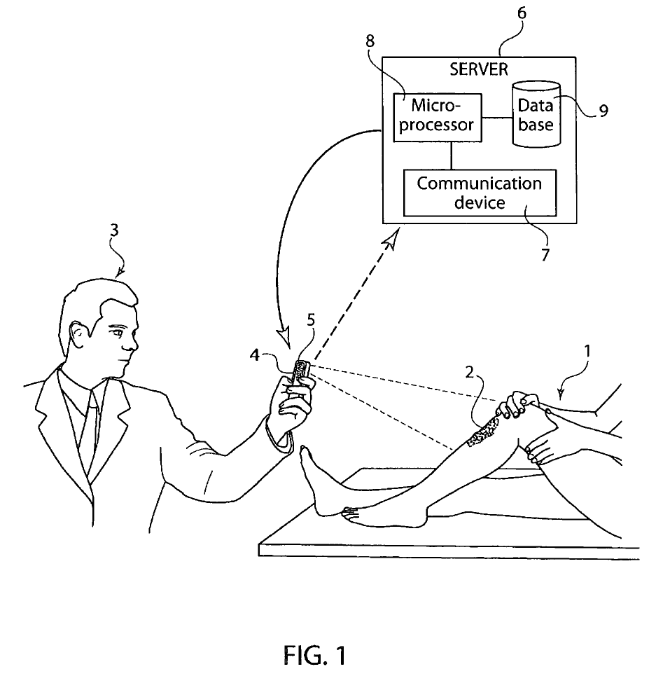

Referring now in detail to the drawings, FIG. 1 shows a patient 1 with an infected wound 2. An individual 3 treating patient 1 has an individual microprocessor such as a mobile smart phone 4 with a built-in camera 5. Camera 5 is adapted to take very high-resolution photographs that can be enlarged to show individual cells and/or bacteria. To treat patient 1, individual 3 takes a photograph of wound 2 using camera 5 in mobile phone 4. The camera 5 could also be connected to a hard-wired microprocessor in a clinical setting. This microprocessor could also be connected with magnification equipment such as an endoscope, which can magnify the diseased area prior to taking the photograph. The photograph is then sent by the individual via text message or email to a server 6 via having a communication device 7, such as a modem. Server 6 is equipped with a microprocessor 8, which can scan the received photograph, and using software installed on server 6, enlarge and enhance the photograph to see the individual cells and bacteria of wound 2. Microprocessor 8 then searches database 9, which contains photographs of many different diseases, to find a match to the photograph sent by individual 3. This search can be conducted with any suitable visual matching software, such as those discussed in U.S. Pat. Nos. 5,911,139 and 7,756,309, the disclosures of which are herein incorporated by reference. Once a match has been found, the microprocessor 8 searches database 9 for a suitable treatment plan, which correlates with the photograph in database 9 that matches the photograph sent by individual 3. Once a treatment plan has been identified, it is sent via communication device 6 to mobile telephone 4 for use in treating patient 1.

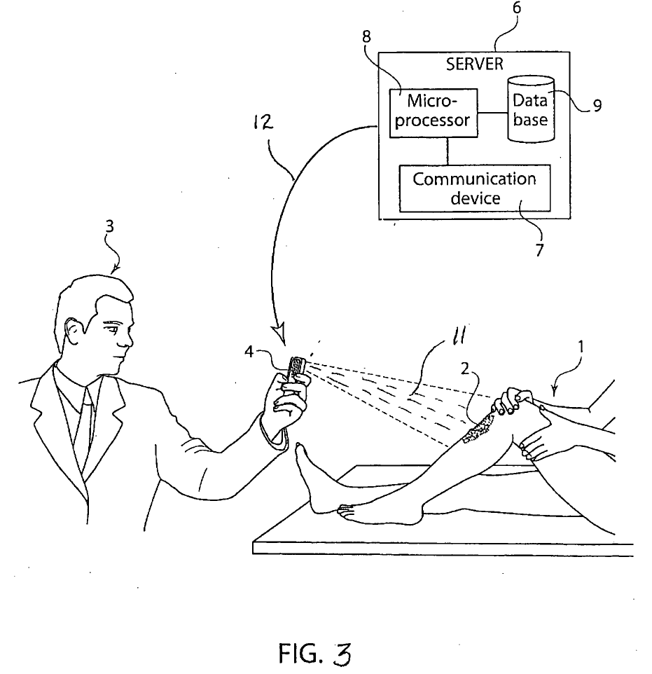

In one embodiment, the treatment plan involves exposing the wound 2 to a wavelength of light that will kill any bacteria in wound 2. As shown in FIG. 3, microprocessor 8 sends a software application 12 to mobile phone 4, which generates such a light beam 11.

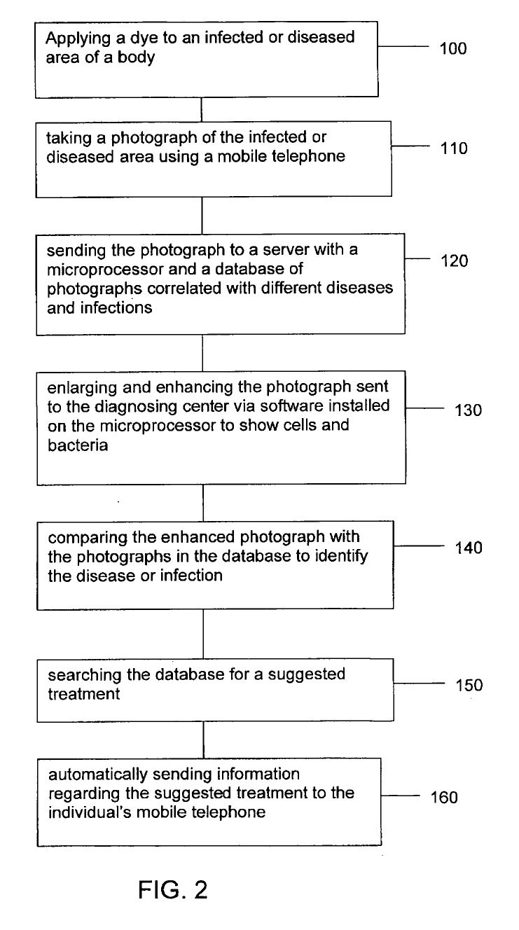

FIG. 2 shows the method steps according to one embodiment of the invention. In step 100, a dye is applied to an infected or diseased area of the body, to enhance the visual resolution of the infected or diseased area. In step 110, a photograph is taken of the infected or diseased area using a smartphone having a built in camera, or using a camera connected to another type of microprocessor, such as a personal computer. Instead of or in addition to the photograph, a video showing the behavior of infected cells could also be taken.

In step 120, the photograph is sent by text message or email to a diagnosing center having a server with a microprocessor and a database of photographs correlated with different diseases and infections. In step 130, the photograph is enlarged and enhanced via software installed on the microprocessor so that individual cells or bacteria can be seen in the enhanced photograph.

In step 140, the enhanced photograph is compared with the photographs in the database to identify the disease or infection, using visual comparison software. In step 150, the microprocessor searches the database for a suggested treatment. In step 160, the microprocessor automatically sends information regarding the suggested treatment to the individual's mobile phone or microprocessor.

Accordingly, while only a few embodiments of the present invention have been shown and described, it is obvious that many changes and modifications may be made thereunto without departing from the spirit and scope of the invention.

What is claimed is:

1. A method for diagnosing and treating infection or disease, comprising:
taking a photograph of an infected or diseased area of a body by an individual using a camera connected to a first microprocessor;
sending the photograph via the first microprocessor to a diagnosing center having a server with a second microprocessor and a database of photographs correlated with different diseases and infections, the photographs showing individual diseased cells or bacteria;
enlarging and enhancing the photograph sent to the diagnosing center via software installed on the second microprocessor so that individual cells or bacteria can be seen in the enhanced photograph;
comparing the enhanced photograph with the photographs in the database to identify the disease or infection, the step of comparing being undertaken by the second microprocessor using visual comparison software;
upon identifying the disease or infection, the second microprocessor searching the database for a suggested treatment; and
automatically sending information regarding the suggested treatment via the second microprocessor to the first microprocessor.

2. The method according to claim 1, further comprising the step of applying a dye to the infected or diseased area prior to the step of taking a photograph.

3. The method according to claim 1, further comprising the step of taking a video of the infected or diseased area and sending the video to the diagnosing center.

4. The method according to claim 1, wherein the information regarding the suggested treatment comprises a prescription for a medication.

5. The method according to claim 1, wherein the information regarding the suggested treatment comprises a software application that when opened emits a light in a wavelength that kills bacteria when the light is aimed at the infected area.

6. The method according to claim 1, wherein the camera and microprocessor are incorporated into a mobile phone.

7. A system for diagnosing and treating infection and disease comprising:
a server having a microprocessor and a searchable database, the database containing a collection of photographs of diseased cells and bacteria, the microprocessor having software that enables a
a communication system connected to the server, said communication system being adapted to receive communications containing photographs from an outside source and send the communications to the server,
wherein the microprocessor is programmed with software that analyzes a photograph in a received communication, compares the photograph to the photographs in the database to find a match, searches for a treatment plan for a disease associated with the match and sends the treatment plan via the communication system to a microprocessor of the outside source.

8. The system according to claim 7, wherein the treatment plan is a prescription for medication, the prescription being stored on the server in the database.

9. The system according to claim 7, wherein the treatment plan is a software application sent to a user's microprocessor, the software application when activated emitting light in a wavelength that is adapted to kill infection.
